(19) United States
(12) Patent Application Publication
(10) Pub. No.: US 2013/0316963 A1
(43) Pub. Date: Nov. 28, 2013

(54) INNATE DEFENCE REGULATORY PEPTIDE COMPOSITIONS FOR TREATMENT OF ARTHRITIS

(71) Applicant: Neeloffer Mookherjee, Winnipeg (CA)

(72) Inventor: Neeloffer Mookherjee, Winnipeg (CA)

(73) Assignee: UNIVERSITY OF MANITOBA, Winnipeg, MB (CA)

(21) Appl. No.: 13/704,971

(22) PCT Filed: Jun. 14, 2011
(86) PCT No.: PCT/CA2011/000703

§ 371 Date: Jul. 25, 2013

Related U.S. Application Data
(60) Provisional application No. 61/355,088, filed on Jun. 15, 2010.

(51) Int. Cl. A61K 38/10

(52) U.S. Cl. CPC A61K 38/10
USPC 514/21.5; 435/375; 435/184

(57) ABSTRACT

A composition for preventing or treating inflammatory arthritis wherein the composition comprises an innate defence regulatory peptide named IDR-1002, said peptide comprising an amino acid sequence listing of VQRWLIVWRIRK, and/or a derivative thereof and/or an analog thereof. Use of the IDR-1002 peptide and/or a derivative thereof and/or an analog or a composition comprising the peptide and/or a derivative and/or analog to modulate the expression and/or function of an inflammatory cytokine and/or a matrix metallopeptidase-3 and/or a cell-signalling pathway.

### TECHNICAL FIELD

The present invention relates to compositions for therapeutic treatment of arthritis. More particularly, this invention relates to compositions comprising an innate defence regulatory peptide IDR-1002 and/or its derivatives and/or analogs for modulating the expression and/or function of an inflammatory cytokine and/or a matrix metallopeptidase-3 and/or a cell-signalling pathway associated with inflammatory arthritis.

### BACKGROUND

Chronic inflammatory arthritis is a debilitating disease which leads to progressive tissue destruction of synovial joints, loss of skeletal function, disability and shortened life expectancy, and is associated with astronomical health care costs. The complex pathophysiology of arthritis involves synergistic interplay between diverse cell populations; primarily fibroblast-like synoviocytes (FLS), immune cells such as macrophages and T-lymphocytes, and their respective pro-inflammatory mediators. A hallmark event in the development of arthritis is the activation of FLS cells in the synovium which results in the production of inflammatory mediators such as cytokines, chemokines, matrix-degrading enzymes such as matrix metallopeptidases, all subsequently contributing to the destruction of articular cartilage and bone. Two critical inflammatory mediators in arthritis are pro-inflammatory cytokines tumour necrosis factor alpha (TNF-α) and interleukin-1 beta (IL-1β). These cytokines induce the production of matrix-degrading enzymes such as matrix metallopeptidase (MMP) and mediate cartilage destruction in synovial joints. However, the genetic regulation and the actual presence of TNF-α and IL-1β in joints appear to be heterogenous. Although the hierarchy of expression of these cytokines in the arthritic synovial microenvironment remain unclear, it has been recently suggested that TNF-α may be the dominant pro-inflammatory cytokine in the acute inflammatory stage of arthritis where as IL-1β is crucial for the propagation of chronic joint inflammation. The heterogeneity of these molecular mechanisms is reinforced by the fact that response to pharmacological treatment varies considerably among patients. For example, there is a lack of clinical response in a substantial number of patients receiving TNF blockers. Also, anti-TNF treatments do not fully control the disease process even in good responders to the treatments suggesting that there are TNF-independent mechanisms involved in the disease process.

Nevertheless, neutralization of critical inflammatory cytokines, especially TNF-α, has been an established principle of current pharmacological therapies in arthritis. Current treatment modalities for chronic inflammatory diseases include systemic blocking of TNF-α, which has shown significant promise in the management of arthritis, psoriasis and inflammatory bowel disease. However, both cytokines TNF-α and IL-1β are essential for efficient immune functions, cellular response to injury, control of infectious agents and neoplasms. Systemic neutralization of these cytokines has been associated with serious side effects resulting in compromised anti-infective immunity, in particular reactivation of tuberculosis and worsening of severe heart failure. Consequently, there are two major disadvantages of employing biologic therapeutic agents for arthritis (and other chronic inflammatory diseases) targeting pro-inflammatory cytokine TNF-α: (i) TNF-independent mechanisms have been implicated in the sustenance of disease-associated inflammation, and (ii) associated increased risk of infections and neoplasm. Therefore, there remains the need for alternate therapeutic strategies for the management of the development and chronicity of the arthritis process that will not compromise efficient immune functioning.

### SUMMARY OF THE INVENTION

This invention is based on the discovery that effective strategies for prevention and/or modulating the symptoms of inflammatory arthritis can be achieved by administering an effective amount of an innate defence regulatory peptide IDR-1002 and/or its derivatives and/or analogs. Some exemplary embodiments of the present invention relate to use of the IDR-1002 peptide and/or its derivatives and/or analogs, for prevention of and/or therapeutic treatment of inflammatory arthritis. Some exemplary embodiments of the present invention relate to use of compositions comprising the IDR-102 peptide and/or its derivatives and/or analogs, for prevention of and/or therapeutic treatment of inflammatory arthritis.

### BRIEF DESCRIPTION OF THE DRAWINGS

The present invention will be described in conjunction with reference to the following drawings, in which:

FIGS. 1A-1C are charts showing the effects of the IDR-1 peptide and the IDR-1002 peptide, according to exemplary embodiments of the present invention, on MMP-3 production in human FLS cells that were previously sensitized with pro-inflammatory cytokines, wherein 1A shows the effects of the peptides on FLS cells stimulated with 10 ng/ml of the IL-1β pro-inflammatory cytokine, 1B shows the effects of the peptides on FLS cells stimulated with 10 ng/ml of the TNF-α pro-inflammatory cytokine, and 1C shows the effects of the peptides on FLS cells stimulated with 10 ng/ml of the IL-1β+TNF-α pro-inflammatory cytokines;

FIGS. 2A-2C are charts showing the effects of treating human FLS cells with the IDR-1002 peptide according to an exemplary embodiment of the present invention, on subsequent sensitivity to pro-inflammatory cytokines, wherein 2A shows the production of MMP-3 by IDR-1002-cultured FLS cells after stimulation with 10 ng/ml of IL-1β pro-inflammatory cytokine for 24 hr, 2B shows the production of MCP-1 by IDR-1002-cultured FLS cells to stimulation with 10 ng/ml of IL-1β for 24 hr, and 2C shows the production of IL-8 by IDR-1002-cultured FLS cells to stimulation with 10 ng/ml of IL-1β for 24 hr;

FIG. 3 is a chart showing the effects of the IDR-1002 peptide on IL-1β-induced NF-κB activation in rabbit synovial fibroblasts;

FIG. 4 is a chart showing the effects of the IDR-1002 peptide on IL-6 production in human FLS cells stimulated with TGF-β1;

FIG. 5 is a chart showing the effects of the IDR-1002 peptide on IL-IRA production in human FLS cells stimulated with IL-1β;

FIG. 6 is a chart showing the effects of the IDR-1002 peptide on transcriptional responses for IL-IRA and SIGIRR in human FLS cells;

FIGS. 7A and 7B are charts showing the effects of the IDR-1002 peptide on the production of pro-inflammatory cytokines TNF-α and IL-1β, respectively, in human macrophage-like THP-1 cells stimulated with pro-inflammatory cytokine IL-32;

FIG. 8 is a micrograph of an immunoblot showing the effects of the IDR-1002 peptide on subsequent JNK activation in human FLS cells;

FIG. 9 is a micrograph of an immunoblot showing the effects of the IDR-1002 peptide on subsequent p38 MAPK activity in human FLS cells;

FIG. 10 is a schematic illustration showing a model for the therapeutic modulation the IDR-1002 peptide of IL-1β-induced responses in synovial fibroblasts.

### DETAILED DESCRIPTION

Unless otherwise defined, all technical and scientific terms used herein have the same meaning as commonly understood by one of ordinary skill in the art to which this invention belongs. In order that the invention herein described may be fully understood, the following terms and definitions are provided herein.

The word “comprise” or variations such as “comprises” or “comprising” will be understood to imply the inclusion of a stated integer or groups of integers but not the exclusion of any other integer or group of integers.

The word “complexed” as used herein means attached together by one or more linkages.

The term “abrogate” as used herein means to suppress and/or interfere with and/or prevent and/or eliminate.

The term “effective amount” as used herein means an amount effective, at dosages and for periods of time necessary to achieve the desired results (e.g. the modulation of collagen synthesis). Effective amounts of a molecule may vary according to factors such as the disease state, age, sex, weight of the animal. Dosage regimes may be adjusted to provide the optimum therapeutic response. For example, several divided doses may be administered daily or the dose may be proportionally reduced as indicated by the exigencies of the therapeutic situation.

The term “subject” as used herein includes all members of the animal kingdom, and specifically includes humans.

The term “a cell” includes a single cell as well as a plurality or population of cells. Administering an agent to a cell includes both in vitro and in vivo administrations.

The term “about” or “approximately” means within 20%, preferably within 10%, and more preferably within 5% of a given value or range.

The term “homologous” in all its grammatical forms and spelling variations refers to the relationship between proteins that possess a “common evolutionary origin,” including homologous proteins from different species. Such proteins (and their encoding genes) have sequence homology, as reflected by their high degree of sequence similarity. This homology is greater than about 75%, greater than about 80%, greater than about 85%. In some cases the homology will be greater than about 90% to 95% or 98%.

“Amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence.

The term “polypeptide” refers to a polymeric compound comprised of covalently linked amino acid residues. Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. A polypeptide of the invention preferably comprises at least about 14 amino acids.

The term “protein” refers to a polypeptide which plays a structural or functional role in a living cell.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

The term “derivative” refers to a product comprising, for example, modifications at the level of the primary structure, such as deletions of one or more residues, substitutions of one or more residues, and/or modifications at the level of one or more residues. The number of residues affected by the modifications may be, for example, from 1, 2 or 3 to 10, 20, or 30 residues. The term derivative also comprises the molecules comprising additional internal or terminal parts, of a peptide nature or otherwise. They may be in particular active parts, markers, amino acids, such as methionine at position −1. The term derivative also comprises the molecules comprising modifications at the level of the tertiary structure (N-terminal end, and the like). The term derivative also comprises sequences homologous to the sequence considered, derived from other cellular sources, and in particular from cells of human origin, or from other organisms, and possessing activity of the same type or of substantially similar type. Such homologous sequences may be obtained by hybridization experiments. The hybridizations may be performed based on nucleic acid libraries, using, as probe, the native sequence or a fragment thereof, under conventional stringency conditions or preferably under high stringency conditions.

Cationic host defence (i.e., antimicrobial) peptides are gene-encoded critical elements of innate immunity that delicately balance inflammatory responses. The initial interests in these molecules were due to assess their anti-microbial properties. However, it has been increasingly suggested that the ability of cationic host defence peptides to protect against pathogenic assault is largely due to their function as innate immune regulators. It has been demonstrated that these naturally occurring molecules exhibit an overall anti-inflammatory effect by suppressing certain pro-inflammatory pathways, and up-regulating or maintaining anti-inflammatory mechanisms. Host defence peptides can modulate activation of the critical inflammatory transcription factor, nuclear factor (NF)-κB, via multiple points of intervention.

The paradox associated with naturally occurring host defence peptides is that they exhibit both anti-inflammatory and pro-inflammatory biological activities. There are some classical pro-inflammatory responses associated with these molecules such as direct chemoattraction of immune cells, induction of chemokines for recruitment and movement of immune cells, differentiation of dendritic cells. These peptides are widely diverse in sequence and structure and thus provide an extensive template for designing short synthetic peptides. More than a thousand different naturally occurring host defence peptides from eukaryotic species have been described. Strategies for designing short synthetic peptides from natural host defence peptides include random mutations of synthetic genes encoding cationic peptides, by robotic synthesis of library of peptides (peptide arrays) using both systematic and random substitutions, and peptide scrambling. Such short synthetic variants of naturally occurring host defence peptides are known as innate defence regulator (IDR) peptides. One such IDR peptide, named IDR-1 was shown to be protective against a variety of infections largely by modulating innate immune responses of the host and up-regulating anti-inflammatory molecular mechanisms (Scott et al., 2007, An anti-infective peptide that selectively modulates the innate immune response. Nat. Biotechnol. 25:456-472). To date, it is not known if there is potential for IDR peptides in limiting the escalation of inflammation in chronic inflammatory or autoimmune disorders, and in particular, those disorders associated with inflammatory arthritis.

We have surprisingly discovered that an effective strategy for modulating the onset and/or symptoms of inflammatory arthritis can be achieved by administering an effective amount of an IDR peptide named IDR-1002 and/or its derivatives and/or analogs. IDR-1002 has the amino acid sequence VQRWLIVWRIRK (SEQ ID: 1). As such, the present invention relates to compositions comprising the IDR-1002 peptide and/or its derivatives exemplified by the amino acid sequence VQRWLIVWRIRK-NH₂ (SEQ ID NO: 2) and/or its analogs for use in the prevention of and/or therapeutic treatment of inflammatory arthritis, and methods of using such compositions for modulating an inflammatory arthritis and related metabolic pathways in a subject.

The data disclosed herein demonstrate that IDR-1002, significantly suppresses IL-1β-induced MMP-3 production in human FLS cells. Furthermore, IDR-1002 suppresses MMP-3 production in the presence of IL-1β, with or without TNF-α, but not that induced by TNF-α alone indicating that IDR-1002 modulates TNF-independent, IL-1β-induced regulatory pathways, and therefore is beneficial in controlling the arthritic disease progression and downstream responses essential for tissue destruction. Employing quantitative proteomics, computational data analysis and further experimental validations, we determined that IDR-1002 alters IL-1β-induced proteome in synovial fibroblasts by modulating the NF-κB, JNK and Hnf-4α pathways.

We also discovered that IDR-1002 suppresses the direct activation of NF-κB in synovial fibroblasts. This discovery is consistent with the paradigm of ‘selective’ immunomodulation of inflammatory responses i.e. suppression of excessive activation of NF-κB in the presence of exogenous infectious/inflammatory stimuli, which result as a consequence of the breakdown of the tightly controlled inflammatory process, while maintaining transient NF-κB activity, overall resulting in balanced inflammatory responses required for anti-infective immunity.

IDR-1002 significantly suppresses IL-1β-induced MMP-3 and also suppresses direct NF-κB activation as well as certain chemokines such as MCP-1. However, IDR-1002 does not abrogate all chemokine production required for efficient functioning of anti-infective immunity. Consequently, the data disclosed herein demonstrates the potential of IDR-1002 and/or its derivatives and/or analogs in selectively altering IL-1β-induced inflammatory responses in human FLS cells resulting in overall balance of inflammation such that tissue destruction is controlled while maintaining essential innate immune functioning.

The studies disclosed herein also demonstrate that IDR-1002 directly suppresses IL-1β-induced JNK activity and P38 MAPK activity. IL-1β-induced JNK activation is known to have a crucial role in the induction of MMPs and subsequent tissue destruction in arthritis. It has been recently demonstrated that IL-1β increases the expression of adhesion molecules in rheumatoid arthritis synovial fibroblasts by activating NF-κB and JNK. Consequently, JNK is a valuable therapeutic target for arthritis. These are the first studies to conclusively demonstrate a direct impact of IDR-1002 on IL-1β-induced JNK activation in human FLS cells. Overall, these studies conclusively demonstrated that the activity of two master inflammatory regulators, NF-κB and JNK, are modulated by IDR-1002 in the presence of the critical inflammatory stimuli IL-1β. Therefore it is clear that IDR-1002 can suppress IL-1β-induced downstream responses that result in increased leukocyte adhesion in the synovial microenvironment, escalation of inflammation and resulting tissue destruction in arthritis. These results taken together lead to the conclusion that IDR-1002 and/or its derivatives and/or analogs and possibly other IDR peptides are useful as therapeutics for arthritis.

Accordingly, some embodiments of the present invention relate to anti-inflammatory compositions comprising IDR-1002 and/or its derivatives and/or its analogs. Some embodiments relate to use of the anti-inflammatory compositions for preventing the occurrence of and/or for modulating the extent of development of inflammatory arthritis.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims. As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth. Unless defined otherwise, all technical and scientific terms used herein have the same meaning as commonly understood by one of ordinary skill in the art to which this invention belongs.

### EXAMPLES

### Cell Isolation and Culture:

Synovial tissues were obtained from patients with OA in accordance to a protocol by the Institutional Review Board at the University of Manitoba. FLS cells were isolated from the synovial tissues following the procedure taught by Kammouni et al. (2007, Regulation of apoptosis in fibroblast-like synoviocytes by the hypoxia-induced Bcl-2 family member Bcl-2/adenovirus E1B 19-kd protein-interacting protein 3. Arthritis Rheum. 56:2854-2863). Briefly, the tissues were digested with 1 mg/ml collagenase and 0.05 mg/ml hyaluronidase (both obtained from Sigma-Aldrich Co., Oakville, ON, Canada) in Hanks' balanced salt solution (Gibco; Invitrogen Canada Inc., Burlington, ON, Canada) for 1-2 hours at 37° C. Cells were washed and cultured in DMEM media (Gibco) supplemented with sodium pyruvate and non-essential amino acids (referred to as complete DMEM media henceforth), containing 10% (v/v) fetal bovine serum (FBS) in a humidified incubator at 37° C. and 10% CO₂. Isolated human FLS cells (ex-vivo) were seeded at 2×10⁴ cells/ml, either 0.5 ml per well in 48-well tissue culture plate, or 3 ml per well in 6-well tissue culture plate as required and cultured in complete DMEM media containing 10% (v/v) FBS overnight. The following day the culture media was changed to complete DMEM containing 1% (v/v) FBS before the addition of the various stimulants. The FLS cells were not used beyond passage five. A rabbit synoviocyte cell line HIG-82 (ATCC® CRL-1832™) was cultured in Ham's F-12 growth medium containing glutamine (GIBCO) supplemented with sodium pyruvate (referred to as complete F-12 media henceforth), containing 10% (v/v) FBS in a humidified incubator at 37° C. and 5% CO₂. Confluent human FLS or HIG-82 cells were trypsinized with 1:3 dilution of 0.5 trypsin-EDTA (Invitrogen) in Hanks' balanced salt solution. Cellular cytotoxicity was evaluated by monitoring the release of lactate dehydrogenase (LDH) employing a colorimetric detection kit (Roche Diagnostics, Laval, QC, Canada).

### Stimulants and Recombinant Cytokines:

Recombinant human cytokines TNF-α and IL-1β were obtained from eBioscience, Inc (San Diego, Calif., USA). IDR-1002 peptide (VQRWLIVWRIRK) was synthesized employing F-moc chemistry at the Nucleic Acid/Protein Synthesis Unit of University of British Columbia, Vancouver, BC, Canada, and IDR-1 peptide having the amino acid sequence KSRIVPAIPVSLL (SEQ ID NO: 3) was obtained from GenScript USA Inc (Piscataway, N.J., USA). The peptides were re-suspended in endotoxin-free water, aliquoted and stored at −20° C.

### Example 1

Tissue culture supernatants were harvested after stimulation of human FLS cells with various cytokines (as indicated) with and without IDR peptides after 24 hr. The supernatants were centrifuged at 1500×g for 7 min to obtain cell-free samples. The samples were aliquoted and stored at −20° C. until further use. Production of MMP-3 in the tissue culture supernatants was monitored using Quantikine® human MMP-3 (total) ELISA kit (Quantikine is a registered trademark of R&D Systems, Inc. Minneapolis, Minn., USA) as per the manufacturer's instructions. Production of pro-inflammatory cytokines IL-1β, IL-6, IL-10, TNF, IL-12p70 and chemokines IL-8, RANTES, MIG, MCP-1, IP-10 in the tissue culture supernatants was determined using preconfigured multiplex BD Cytometric Bead Array (CBA) human inflammation and chemokine kits respectively, employing the FACS Calibur flow cytometer (BD Biosciences, Mississauga, ON, Canada) as per the manufacture's instructions. The concentration of the cytokines or chemokines in the tissue culture supernatants was evaluated by establishing a standard curve with serial dilutions of the recombinant human cytokines or chemokines as required.

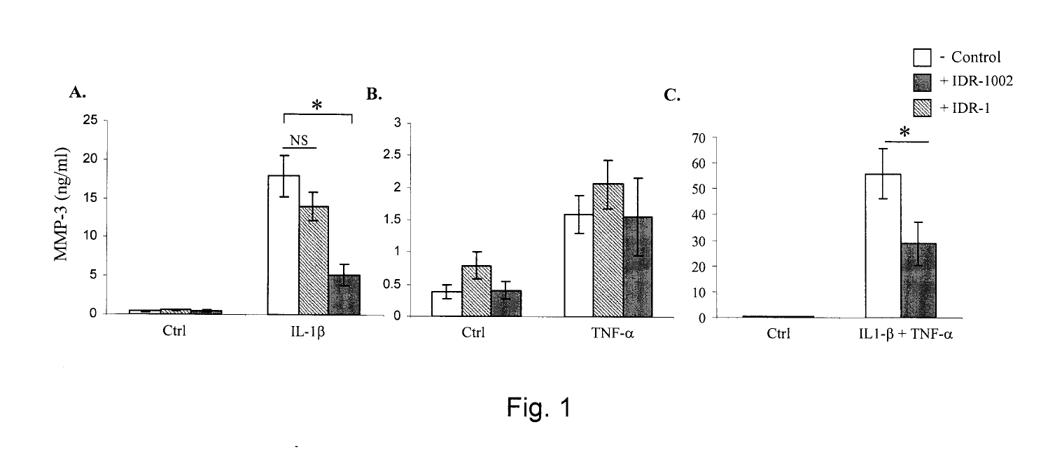

Inflammatory cytokines TNF-α and IL-1β stimulate cells types such as FLS, chondrocytes and macrophages resulting in the production of MMP-3 in arthritic joints. The elevated level of MMP-3 is known to cause cartilage and bone destruction. In this study we evaluated the impact of IDR peptides on TNF-α and IL-1β-induced MMP-3 production. Human FLS cells (ex-vivo) were stimulated with pro-inflammatory cytokines either TNF-α or IL-1β (10 ng/ml) or the combination of the two cytokines, in the presence and absence of IDR peptides either IDR-1002 (100 μg/ml) or IDR-1 (200 μg/ml). The peptides were added at the time of cytokine stimulation. The peptides were not cytotoxic to the FLS cells in the presence and absence of cytokine stimulation, as determined by monitoring the tissue culture supernatants for the release of LDH after 24 hr of stimulation (data not shown). Tissue culture supernatants were monitored after 24 hr of stimulation for MMP-3 production by ELISA. IDR-1002 significantly (p<0.05) suppressed IL-1β-induced MMP-3 by 70±8% (FIG. 1A), but not TNF-α-induced MMP-3 (FIG. 1B) in human FLS cells. IDR-1002 also significantly (p<0.05) suppressed MMP-3 production induced in the presence cytomix (TNF-α+IL-1β, 10 ng/ml each) by 56±10% (FIG. 1C). IDR-1002 by itself did not induce MMP-3 production above the background amount observed in un-stimulated control FLS cells (FIG. 1A). In contrast, the IDR-1 peptide did not suppress either IL-1β or TNF-α-induced MMP-3 production in FLS cells (FIGS. 1A and 1B respectively). Results shown are an average of at least three independent biological experiments performed with cells isolated from synovial tissues obtained from independent donors±standard error (*p<0.05, **p<0.01).

To evaluate whether IDR-1002 alone could induce inflammatory responses, human FLS cells were stimulated with IDR-1002 (100 μg/ml) for 24 hr. BD Cytometric Bead Array (CBA) preconfigured human inflammation (IL-8, IL-1β, IL-6, IL-10, TNF, IL-12p70) kit was used to evaluate protein secretion of the various analytes in the tissue culture supernatants employing FACS Calibur flow cytometer. IDR-1002 did not induce the release of pro-inflammatory cytokines TNF-α, IL-1β or IL6 above the background levels detected in un-stimulated control cells (data not shown), and IL-10 and IL-12p70 could not be detected in the supernatants by flow cytometry.

Taken together, these results demonstrated that IDR peptide 1002 significantly suppressed IL-1β- or cytomix (IL-1β+TNF-α)—induced MMP-3 (known to mediate cartilage destruction and facilitate the pathogenesis of arthritis) and did not induce pro-inflammatory cytokines in human FLS cells.

### Example 2

Amine-modifying iTRAQ® reagents multiplex kit (iTRAQ is a registered trademark of AB Sciex PTE Ltd., Foster City, Calif., USA) was employed for relative quantitation of proteins in human FLS cells stimulated with IL-1β in the presence and absence of IDR-1002 compared to un-stimulated (control) cells. Human FLS cells (2×10⁴/ml) were seeded in a total volume of 3 ml per well in a 6-well tissue culture plate in complete DMEM media containing 10% FCS. The cells were allowed to adhere overnight. Following day the media was changed to 3 ml complete DMEM containing 1% FBS per well. The cells were either un-stimulated or treated with IL-1β (10 ng/ml) in the presence or absence of IDR-1002. The peptide (100 μg/ml) was added 45 min prior to stimulation with IL-1β. After 24 hr of stimulation, the cells were washed with cold PBS and lysed in 250 μl of buffer containing 10 mM Tris pH 7.5, 150 mM NaCl, 2 mM EDTA, 1% NP-40 and protease inhibitor cocktail (Sigma-Aldrich), on ice for 30 min with intermittent vortexing. Cells were centrifuged at 10,000×g for 10 min at 4° C. Total protein content was estimated in each cell lysate employing micro BCA assay (Pierce; Thermo Scientific, Rockford, Ill., USA) with a bovine serum albumin (BSA) (Sigma-Aldrich) standard curve. The samples were precipitated with acetone at −20° C. overnight. Proteins were dissolved in 20 ul of iTRAQ® dissolution buffer and were further processed following the manufacturer's instructions. Briefly, proteins were reduced and the cysteines blocked using the reagents in kit, followed by digestion of the protein samples with provided trypsin solution overnight at 37° C. The trypsin-digested protein samples were labelled with the iTRAQ® isobaric tags as follows: Un-stimulated (control) samples were labelled with iTRAQ® isobaric tag 115, IL-1β-stimulated sample with tag 116, and the isobaric tag 117 was used for labelling the sample obtained from cells treated with IL-1β in the presence of IDR-1002. The contents from each of the iTRAQ® reagent-labelled sample was combined together in 1:1 ratio and processed for nanoflow liquid chromatography coupled to tandem mass spectrometry.

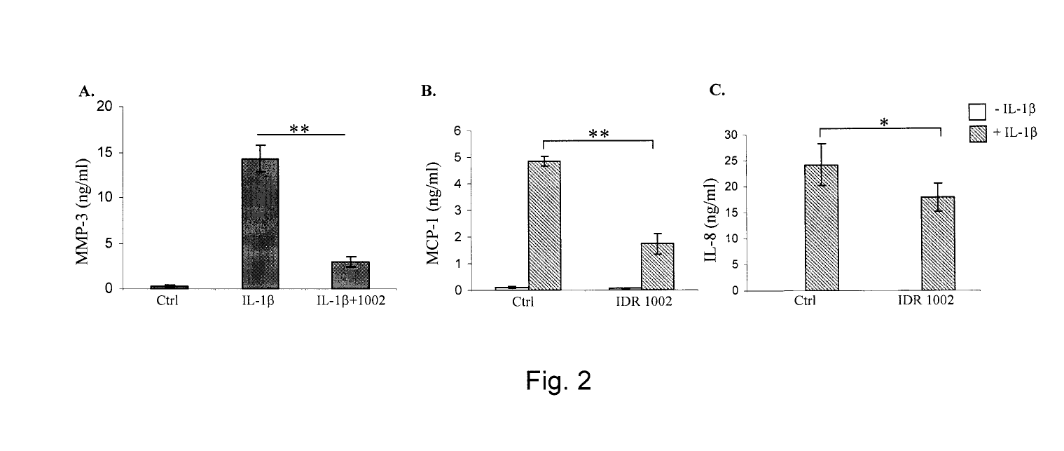

IL-1β-induced protein profiles (proteome) in the presence and absence of IDR-1002 were evaluated using quantitative proteomics iTRAQ® tools employing different isobaric tags. Human FLS cells were treated with IDR-1002 (100 μg/ml) for 45 min prior to IL-1β (10 ng/ml) stimulation for 24 hr. Tissue culture supernatants were monitored for protein production of MMP-3 and chemokines IL-8 and MCP-1 production, in order to evaluate the validity of the assay before processing the cell lysates for quantitative proteomic evaluation. MMP-3 production was monitored in the tissue culture supernatants by ELISA, and chemokines IL-8 and MCP-1 production were evaluated by BD preconfigured human chemokine Cytometric Bead Array. IDR-1002 significantly (p<0.01) suppressed IL-1β-induced MMP-3 by 80% (FIG. 2A) and chemokine MCP-1 production >60% (FIG. 2B) in human FLS cells. However, IL-1β-induced chemokine IL-8 production (FIG. 2C) was modestly suppressed (by 20%, p<0.05) in the presence of IDR-1002 in FLS cells. This is consistent with previous studies demonstrating that host defence peptides can selectively modulate overall inflammatory processes without abrogating chemokine responses that are required for cell movement and recruitment essential to combat infectious assault.

The FLS cell lysates obtained after stimulation with IL-1β in the presence and absence of IDR-1002 were processed for iTRAQ® labelling using three different isobaric tags. Three independent LC-MS/MS runs were performed on iTRAQ®-labelled samples from three independent donors. Protein candidates were selected only if they were detected in at least two out of the three independent biological experiments with 95% confidence. Proteins were defined to be induced if the relative ratios compared to the un-stimulated controls (fold change) were ≧mean±1.3 SD. Based on these selection criteria, 48 proteins were identified to be induced in the human FLS cells up on stimulation with IL-1β, of which 11 proteins were suppressed by at least 20% in the presence of IDR-1002 (Table 1).

TABLE 1
Effects of IDR-1002 on IL-1β-induced proteins.

| | | IL-1 | IL1 + 1002 | |
|---|---|---|---|---|
| | REFSEQ_{—} | Fold | Fold | % Decrease |
| Gene Name | PROTEIN | Change | Change | With 1002 |
| Adenylosuccinate synthase | NP_001117 | 2.45 | 0 | 100 |
| Aminopeptidase-like-1 | NP_078939 | 1.37 | 0 | 100 |
| Ribosomal protein L27A | NP_000981 | 1.37 | 0.8 | 41.6 |
| Kynureninase (L-Kynurenine hydrolase) | NP_001028170 | 4.05 | 2.52 | 37.8 |
| Aldo-keto reductase family 1, Member C3 | NP_003730 | 1.52 | 0.95 | 37.5 |
| Amyloid beta (A4) precursor protein | NP_958817 | 1.85 | 1.27 | 31.4 |
| Annexin A5 | NP_001145 | 1.4 | 1.06 | 24.3 |
| Carboxylesterase 1 (monocyte/macrophage serine | NP_001020365 | 1.41 | 1.07 | 24.1 |
| esterase 1) | | | | |
| Aldo-keto reductase family 1, Member B1 | NP_001619 | 2.18 | 1.67 | 23.4 |
| Cytochrome P450, family 1, subfamily B, | NP_000095 | 1.46 | 1.12 | 23.3 |
| polypeptide 1 | | | | |
| Actin, beta | NP_001092 | 1.4 | 1.1 | 21.4 |
| Interleukin 1 receptor antagonist | NP_776213 | 2.2 | 1.86 | 15.5 |
| Protein kinase C, cAMP-dependent, regulatory, | NP_004148 | 2.16 | 1.84 | 14.8 |
| type II, alpha | | | | |
| Chromatin modifying protein 1B | NP_065145 | 1.38 | 1.18 | 14.5 |
| MARCKS-like 1 | NP_075385 | 1.4 | 1.22 | 12.9 |
| Chitinase 3-like 1 (cartilage glycoprotein-39) | NP_001267 | 2.37 | 2.14 | 9.7 |
| Chromobox homolog 5 (HP1 alpha homolog, | NP_036249 | 1.47 | 1.33 | 9.5 |
| Drosophila) | | | | |
| Branched chain aminotransferase 1, cytosolic | NP_005495 | 1.39 | 1.26 | 9.4 |
| Small GTP-binding protein | XP_950630 | 1.77 | 1.64 | 7.3 |
| N-acetylglucosamine kinase | NP_060037 | 1.46 | 1.37 | 6.2 |
| Thioredoxin reductase 2 | NP_006431 | 1.42 | 1.35 | 4.9 |
| Solute carrier family 39 (Zinc transporter), | NP_056174 | 1.78 | 1.72 | 3.4 |
| member 14 | | | | |
| CD82 antigen | NP_001020015 | 1.9 | 1.87 | 1.6 |
| Superoxide dismutase 2, mitochondrial | NP_001019637 | 1.96 | 1.98 | N/A |
| RAS suppressor protein 1 | NP_036557 | 1.37 | 1.43 | N/A |
| Aldo-Keto reductase family 1, member C1 | NP_001344 | 1.95 | 2.06 | N/A |
| Drebrin 1 | NP_004386 | 1.41 | 1.5 | N/A |
| Pre-B-cell colony enhancing factor 1 | NP_877591 | 1.54 | 1.7 | N/A |
| N-acetyl neuraminic acid synthase (sialic acid | NP_061819 | 1.47 | 1.63 | N/A |
| synthase) | | | | |
| Interleukin 1 family, member 5 (Delta) | NP_775262 | 2.01 | 2.27 | N/A |
| Glutamine-fructose-6-phosphate transaminase 1 | NP_002047 | 1.44 | 1.63 | N/A |
| Major histocompatibility complex, Class IB | NP_005505 | 1.36 | 1.57 | N/A |
| Glutamine-fructose-6-phosphate transaminase 2 | NP_005101 | 1.46 | 1.71 | N/A |
| Annexin A7 | NP_001147 | 1.53 | 1.8 | N/A |
| Intercellular adhesion molecule 1 (CD54) | NP_000192 | 1.47 | 1.73 | N/A |
| Syntaxin 7 | NP_003560 | 1.43 | 1.72 | N/A |
| Vesicle docking protein P115 | NP_003706 | 1.42 | 1.71 | N/A |
| RHO family GTPase 3 | NP_005159 | 1.55 | 1.87 | N/A |
| Ribosomal protein L23 | NP_000969 | 1.43 | 1.74 | N/A |
| Tubulin, alpha, ubiquitous | NP_116093 | 1.37 | 1.71 | N/A |
| Kinesin 2 | NP_005543 | 1.64 | 2.09 | N/A |
| Similar to metallothionein 1G | XP_497514 | 1.78 | 2.27 | N/A |
| ADP-ribosylation factor 4 | NP_001651 | 1.38 | 1.76 | N/A |
| Zinc metallopeptidase (STE24 homolog, yeast) | NP_005848 | 1.55 | 2.34 | N/A |
| Inter-alpha (globulin) inhibitor H2 | NP_002207 | 1.46 | 2.32 | N/A |
| Metalllothionein 1A (functional) | NP_005937 | 1.51 | 2.4 | N/A |
| Arylacetamide deactylase-like 1 | NP_065843 | 1.53 | 2.59 | N/A |
| Tumor necrosis factor, alpha-induced protein 6 | NP_009046 | 2 | 4.98 | N/A |

In order to discover immunity-related modules or pathways that may be involved in the alteration of IL-1β-induced responses in the presence of IDR-1002, we took a network-based approach. The 11 identified IL-1β-induced protein candidates that were found to be suppressed in the presence of the IDR-1002 (Table 1) were submitted to InnateDB biomolecular interaction database. The InnateDB platform facilitates Systems-level analysis of mammalian immune genes and protein products. This database was used to identify direct interactions between the 11 identified protein candidates in this study and any known immunity-related proteins. Computational network analysis demonstrated that several members of both Nuclear Factor (NF)-κB and mitogen activated protein kinase-8 (MAPK8) pathways were direct interactors of the 11 protein candidates in this study (Table 2). Previous studied have conclusively demonstrated that both NF-κB and MAPK-mediated pathways are activated on stimulation with cytokine IL-1β. Four of the 12 identified proteins participated in binary interactions with candidates known to participate in NF-κB activation. These included (i) IκBκE, known to activate NF-κB via TNF-receptor-associated factor (TRAF)-2, (ii) TRAF-6, a regulator of the NF-κB pathway, suggested to play a critical role in human autoimmune diseases including arthritis, (iii) TNF-receptor superfamily member TNFRSF21, shown to activate both NF-κB and MAPK-8 pathways, and (iv) the enzyme encoded by the gene MAP3K14, which activates NF-κB via TRAF-2. Similarly, the network-based analysis in this study demonstrated the likelihood of modulation of IL-1-β-induced MAPK-8-mediated pathway, also known as the c-Jun N-terminal kinases (INK) pathway. Several members of the INK pathway, namely MAPK8, MAPK8IP1 and TNFRSF21 were identified in the interaction protein network of IL-1β-induced candidates that were suppressed by IDR-1002 in human FLS cells (Table 2).

TABLE 2

| | | | IL1 + | | | % |
|---|---|---|---|---|---|---|
| | | IL1 | IDR-1002 | | | Decrease |
| | | Fold | Fold | | Interaction | With |
| RefSeq | Name | Change | Change | Interaction | Type | IDR-1002 |
| NP_001117 | ADSS | 2.45 | 0 | ADSS interacts with IKBKE and with HLA-B | physical | 100.00 |
| | | | | | association | |
| NP_001117 | ADSS | 2.45 | 0 | Transcription factor HNF4A binds with ADSS gene | unspecified | 100.00 |
| NP_078939 | NPEPL1 | 1.37 | 0 | No interactions | | 100.00 |
| NP_000981 | RPL27A | 1.37 | 0.8 | RPL27A interacts with MAP3K14 | physical | 41.61 |
| | | | | | association | |
| NP_000981 | RPL27A | 1.37 | 0.8 | ACTB::ADSL; TNRC6B, AGK, ARF4, ATP5C1, | unspecified | 41.61 |
| | | | | ATP5I, CCT5, CDIPT, | | |
| | | | | DBT, DNAJA1, DNAJA2, DNAJB11, EEF1A1, | | |
| | | | | EIF2C2, EIF2C3, EIF2C4, EIF4B, EMD, GALK1, | | |
| | | | | HIST1H2AB, HNRNPC, HSP90AA1, HSP90AB1, | | |
| | | | | HSPA1B, HSPA5, HSPA8, IDBG-12906, | | |
| | | | | IGF2BP1, IPO8, JAK1, MYCBP, PABPC1, | | |
| | | | | PABPC4, PGAM5, PRDX1, PRMT5, PTGES3, | | |
| | | | | PTS, QPCTL, RBM10, RPL11, RPL12, RPL23, | | |
| | | | | RPL24, RPL27, RPL27A, RPL35, RPL38, RPL8, | | |
| | | | | RPS10L, RPS12, RPS18, RPS25, RPS26, RPS3A, | | |
| | | | | RPS5, RPS9, SLC25A1, SLC25A10, SLC25A13, | | |
| | | | | SLC25A22, SLC25A3, SLC25A5, SNRPD2, | | |
| | | | | SSBP1, SUCLA2, TNRC6A, TRIM21, TUBA1A, | | |
| | | | | TUBB, TUBB2C, TUFM, TUT1, WDR77, YBX1 | | |
| | | | | (complex) | | |
| NP_000981 | RPL27A | 1.37 | 0.8 | ATP5C1::C3orf26, COPA, DDOST, DDX20, | unspecified | 41.61 |
| | | | | DDX39, DHCR7, DHX15, | | |
| | | | | DHX30, DHX36, DHX9, EEF1A1, EIF2C2, EPRS, | | |
| | | | | FBL, GEMIN4, GNL3, HNRNPC, HNRNPU, | | |
| | | | | HRNR, ILF2, MRPS22, MRPS27, PABPC1, PHB, | | |
| | | | | PHB2, PRMT5, PTCD3, RBM10, RNF149, RPL11, | | |
| | | | | RPL13A, RPL23A, RPL24, RPL26, RPL27A, | | |
| | | | | RPL27A, RPL29, RPL3, RPL31, RPL35, RPL4, | | |
| | | | | RPL6, RPL8, RPS15A, RPS18, RPS2, RPS26P25, | | |
| | | | | RPS3, RPS3A, RPS4X, RPS5, RPS6, RPS8, RPS9, | | |
| | | | | RUVBL2, SF3B1, SF3B2, SF3B3, SLC25A3, | | |
| | | | | SLC25A6, SLC4A5, SNORD58B, SYNE1, UBA52, | | |
| | | | | UBA52, XRCC6, YBX1 (complex) | | |
| NP_001028170 | KYNU | 4.05 | 2.52 | No interactions | | 37.78 |
| NP_003730 | AKR1C3 | 1.52 | 0.95 | AKR1C3 interacts with MAGEA11, RIF1, AC1N1, | physical | 37.50 |
| | | | | C1orf103 | association | |
| NP_003730 | AKR1C3 | 1.52 | 0.95 | AKR1C3 interacts with MAGEA11, ZHX1, | unspecified | 37.50 |
| | | | | UBE2W | | |
| NP_003730 | AKR1C3 | 1.52 | 0.95 | Transcription factor HNF1A binds with AKR1C3 | unspecified | 37.50 |
| | | | | gene | | |
| NP_958817 | APP | 1.85 | 1.27 | Cleavage reaction involving APP and CTSD | cleavage | 31.35 |
| | | | | | reaction | |
| NP_958817 | APP | 1.85 | 1.27 | Cleavage reaction involving APP and CASP3; | cleavage | 31.35 |
| | | | | CASP6; CASP8 | reaction | |
| NP_958817 | APP | 1.85 | 1.27 | Cleavage reaction involving ADAM17 and APP | cleavage | 31.35 |
| | | | | | reaction | |
| NP_958817 | APP | 1.85 | 1.27 | Colocalization of APP and CHRNA7 | colocalization | 31.35 |
| NP_958817 | APP | 1.85 | 1.27 | Colocalization of APP and APP | colocalization | 31.35 |
| NP_958817 | APP | 1.85 | 1.27 | Colocalization of APP and MAPK8IP1 | colocalization | 31.35 |
| NP_958817 | APP | 1.85 | 1.27 | Colocalization of APP and MAPT | colocalization | 31.35 |
| NP_958817 | APP | 1.85 | 1.27 | Colocalization of APP and PIN1 | colocalization | 31.35 |
| NP_958817 | APP | 1.85 | 1.27 | Colocalization of APP and BACE1 | colocalization | 31.35 |
| NP_958817 | APP | 1.85 | 1.27 | APP, MAPK8, MAPK8IP1 (complex) | complex | 31.35 |
| | | | | APP, MAP3K11, MAPK8IP1 (complex) | assembly | |
| NP_958817 | APP | 1.85 | 1.27 | APP (complex) | direct | 31.35 |
| | | | | | interaction | |
| NP_958817 | APP | 1.85 | 1.27 | APP interacts with TNFRSF21, MAPT, | direct | 31.35 |
| | | | | NGFR | interaction | |
| NP_958817 | APP | 1.85 | 1.27 | Phosphorylation of APP by MAPK8 | phosphorylation | 31.35 |
| NP_958817 | APP | 1.85 | 1.27 | Phosphorylation of APP by Ab11 | phosphorylation | 31.35 |
| | | | | | reaction | |
| NP_958817 | APP | 1.85 | 1.27 | APP, GSK3A, MAPT (complex) | phosphorylation | 31.35 |
| | | | | | reaction | |
| NP_958817 | APP | 1.85 | 1.27 | APP interacts with APOA1, APBB1, APBB2, | physical | 31.35 |
| | | | | APBB3, APBA1, SHC1, SHC3, TGFB2, TGFB1, | association | |
| | | | | CHRNA7, TP53BP2, MAPK8IP1, PRNP, | | |
| | | | | HSD17B10, GRB2, APOE, ACHE, TTR, A2M, | | |
| | | | | FLOT1, Slc5a7, PSEN1, PSEN2, NF1, PDIA3, | | |
| | | | | PIN1, TUBB, NSF, STXBP1, DNM1, DNAH1, | | |
| | | | | HSP90AA1, HSPA8, CRYAB, PPIA, SPTAN1, | | |
| | | | | ACTB, NEFL, MBP, GFAP, YWHAZ, UCHL1, | | |
| | | | | PGAM1, MAP3K5, SMUG1 | | |
| NP_958817 | APP | 1.85 | 1.27 | APP physically interacts with MAPK8IP1 | physical | 31.35 |
| | | | | | interaction | |
| NP_958817 | APP | 1.85 | 1.27 | APP physically interacts with XIAP | physical | 31.35 |
| | | | | | interaction | |
| NP_958817 | APP | 1.85 | 1.27 | APP interacts with KLK6 | protein | 31.35 |
| | | | | | cleavage | |
| NP_958817 | APP | 1.85 | 1.27 | APP interacts with BACE1 | protein | 31.35 |
| | | | | | cleavage | |
| NP_000095 | CYP1B1 | 1.5 | 1.1 | CYP1B1 interacts with SAE1 | physical | 26.67 |
| | | | | | association | |
| NP_000095 | CYP1B1 | 1.5 | 1.1 | Transcription factor HNF4A binds with CYP1B1 | unspecified | 26.67 |
| | | | | gene | | |
| NP_001145 | ANXA5 | 1.4 | 1.06 | ANXA5 interacts with FDFT1, SUPT4H1, EIF4G1, | physical | 24.29 |
| | | | | CFTR, IFNGR2 | association | |
| NP_001145 | ANXA5 | 1.4 | 1.06 | ANXA5 interacts with ITGB5 | unspecified | 24.29 |
| NP_001145 | ANXA5 | 1.4 | 1.06 | Transcription factor HNF4A binds with ANXA5 | unspecified | 24.29 |
| | | | | gene | | |
| NP_001020365 | CES1 | 1.41 | 1.07 | CES1 interacts with CES1, GUSB | unspecified | 24.11 |
| NP_001619 | AKR1B1 | 2.18 | 1.67 | AKR1B1 interacts with IKBKE, TRAF6, HLA-B, | physical | 23.39 |
| | | | | SMAD1, TFE3, | association | |
| | | | | DSP, MCC, DST, PAX7. CSMD1, ZNF253, VHL | | |
| NP_001619 | AKR1B1 | 2.18 | 1.67 | Transcription factor HNF4A binds with AKR1B1 | unspecified | 23.39 |
| | | | | gene | | |
| NP_001092 | ACTB | 1.4 | 1.1 | A2M::ACTB, ALPP, APOD, ARL8B, ASAH1, | colocalization | 21.43 |
| | | | | ATP5A1, ATP5B, ATP6V0D1, ATP6V1A, | | |
| | | | | ATP6V1B2, AZU1, CAPN6, CKMT1A, CTSG, | | |
| | | | | CYP11A1, CYP19A1, DDOST, DLST, GAPDH, | | |
| | | | | GBA, GLB1, GUSB, HSPA5, HSPD1, MAOA, | | |
| | | | | MPO, PRTN3, SCARB2, SLC25A5, SLC25A6, | | |
| | | | | STS, TPP1, VDAC1 (complex) | | |
| NP_001092 | ACTB | 1.4 | 1.1 | Colocalization of ACTB and IDBG-44570 | colocalization | 21.43 |
| NP_001092 | ACTB | 1.4 | 1.1 | Colocalization of ACTB and RP23-157O10.7 | colocalization | 21.43 |
| NP_001092 | ACTB | 1.4 | 1.1 | Colocalization of ACTB and FBL | colocalization | 21.43 |
| NP_001092 | ACTB | 1.4 | 1.1 | Colocalization of ACTB and Pkd1 | colocalization | 21.43 |
| NP_001092 | ACTB | 1.4 | 1.1 | Colocalization of ACTB and MMP14 | colocalization | 21.43 |
| NP_001092 | ACTB | 1.4 | 1.1 | Colocalization of ACTB and BCAR1 | colocalization | 21.43 |
| NP_001092 | ACTB | 1.4 | 1.1 | ACTB and Sorbs1 | direct | 21.43 |
| | | | | | interaction | |
| NP_001092 | ACTB | 1.4 | 1.1 | ACTB and NCF1C | direct | 21.43 |
| | | | | | interaction | |
| NP_001092 | ACTB | 1.4 | 1.1 | ACTB (complex) | direct | 21.43 |
| | | | | | interaction | |
| NP_001092 | ACTB | 1.4 | 1.1 | ACTB interacts with CFL1, CFL2, ACTB, | physical | 21.43 |
| | | | | ACTG1, DSTN, AR | association | |
| NP_001092 | ACTB | 1.4 | 1.1 | ACTB::ANXA1, ANXA2, ANXA6, ATP5A1, CD4, | physical | 21.43 |
| | | | | DDX3X, DDX5, DHRS2, | association | |
| | | | | EEF1B2, EIF3B, EIF3C, EIF3D, EIF3E, EIF3I, | | |
| | | | | EIF3K, EIF3M, EIF4A2, ENO1, FARSB, GAPDH, | | |
| | | | | GNAI2, GNB2L1, HIST1H2BI, HNRNPA1, | | |
| | | | | HNRNPA2B1, HNRNPD, HNRNPH1, HSP90AA1, | | |
| | | | | HSP90AB1, HSPA8, HSPD1, KPNB1, LCK, | | |
| | | | | LRPPRC, MME, MYL12A, MYL6, NCL, NPM1, | | |
| | | | | PHB, RAN, RPL11, RPL18, RPL22, RPL7, | | |
| | | | | RPL7A, RPLP0, RPLP1, RPS10, RPS12, RPS13, | | |
| | | | | RPS18, RPS19, RPS24, RPS3A, RPS4X, RPS7, | | |
| | | | | RPS8, RPS9, RPSA, SSRP1, TNPO1, TUBA1A, | | |
| | | | | TUBB, UBC, VDAC1, VDAC2, VDAC3, VIM, | | |
| | | | | YBX1 (complex) | | |
| NP_001092 | ACTB | 1.4 | 1.1 | ACTB interacts with SMAD3, SMAD9, MDM2, | physical | 21.43 |
| | | | | NSMAF, ATF7IP, TJP1, YWHAZ, BBS1, BBS4, | association | |
| | | | | APP | | |
| NP_001092 | ACTB | 1.4 | 1.1 | ACTB physically interacts with TSC1 | physical | 21.43 |
| | | | | | interaction | |
| NP_001092 | ACTB | 1.4 | 1.1 | ACTB::ACTL6A, KAT5, RUVBL1, RUVBL2, | unspecified | 21.43 |
| | | | | TRRAP (complex) | | |
| NP_001092 | ACTB | 1.4 | 1.1 | ACLY::ACTB, ACTG1, ACTN4, CDK6, | unspecified | 21.43 |
| | | | | CDKN2A, EEF2, EPHA3, | | |
| | | | | GAPDH, HNRNPA2B1, HNRNPC, HSP90AA1, | | |
| | | | | HSP90AB1, HSPA4, HSPA8, HSPA9, MCM6, | | |
| | | | | MMRN1, MTR, MYL12A, PCNA, PDCD6; AHRR, | | |
| | | | | RIN2, RUVBL2, SNRPA, SNRPB, TUBA1A, | | |
| | | | | TUBA1C, TUBB, TUBB2C, UBE4B, USP26 | | |
| | | | | (complex) | | |

Apart from the identification of the NF-κB and JNK family of proteins, computation analysis of the IL-1β-induced interaction network modulated by IDR-1002 demonstrated that the transcription factor hepatocyte nuclear factor (HNF)-4α had binding sites to the genes encoding 4 out of the 12 identified proteins. Taken together, the network-based interrogation of the IL-1β-induced proteins that were identified to be suppressed in the presence of the IDR-1002 suggested the involvement of three regulatory pathways; (a) NF-κB, (b) MAPK8/JNK activity, and (c) regulation by HNF transcription factors namely HNF-4α.

### Example 3

Rabbit synoviocyte HIG-82 cells were transiently transfected with pNFκB-MetLuc2-Reporter Vector (Clontech laboratories Inc., Mountain View, Calif., USA) or the provided control vector as per the manufacturer's instructions. Various stimulants were added to the transfected cells in culture media containing 1% (v/v) FBS. The cells were stimulated with recombinant human IL-1β in the presence and absence of IDR-1002 for 6 hr. The peptide was added either 45 min prior to, or at the time of cytokine stimulation. The activation of NF-κB was monitored by employing the Ready-To-Glow Secreted NF-κB Luciferase Reporter Assay (Clontech) as per the manufacturer's instructions.

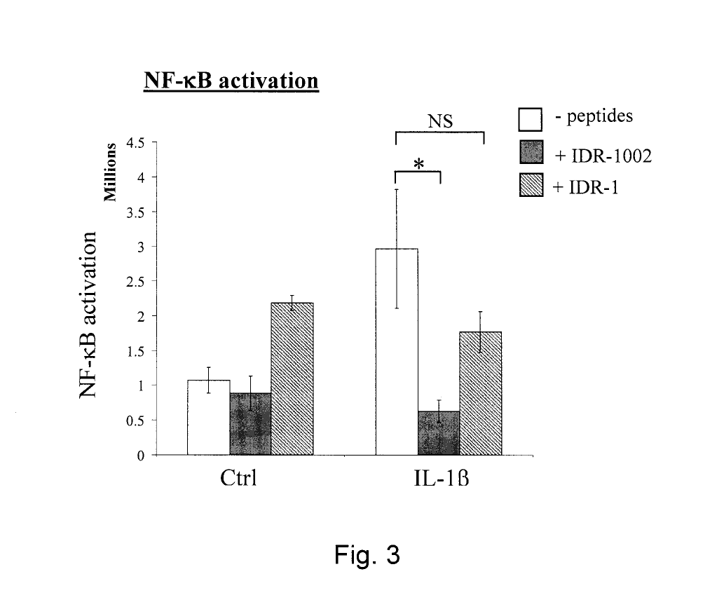

Transcription factor NF-κB is central to the destructive effects associated with the escalation and sustenance of inflammatory responses pivotal in chronic inflammatory diseases including arthritis. Quantitative proteomics evaluation using iTRAQ® labelling pointed to the possibility that the peptide IDR-1002 altered IL-1β-induced NF-κB activation. Therefore, this study further evaluated the impact of IDR-1002 on IL-1β-induced direct activation of NF-κB in synovial fibroblasts. A rabbit synovial fibroblast cell line (HIG82) was transiently transfected with pNFκB-MetLuc2-Reporter Vector (Clontech). The cells were stimulated with IL-1β (10 ng/ml each), in the presence and absence of IDR peptide 1002 (100 μg/ml). The activation of NF-κB was monitored by employing the Ready-To-Glow Secreted NF-κB Luciferase Reporter Assay (Clontech) as per the manufacturer's instructions. IDR-1002 significantly (p<0.05) suppressed IL-1β-induced activation of NF-κB by greater than 70% in rabbit FLS cells (FIG. 3).

### Example 4

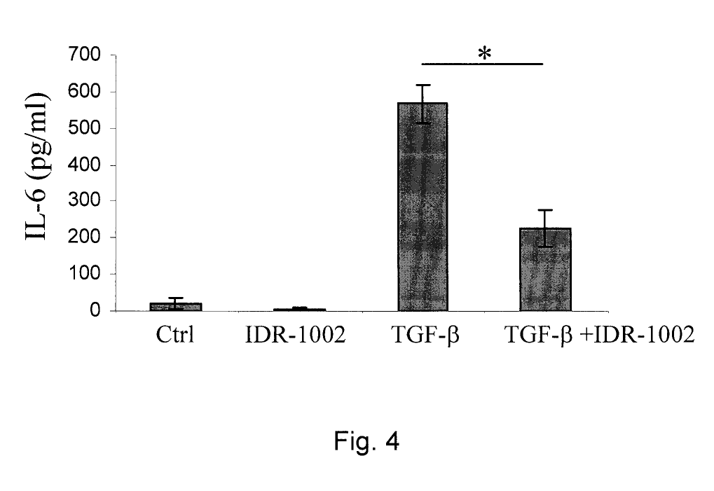

Tissue culture supernatants were harvested from human FLS cells after stimulation for 24 hr with TGF-β with and without IDR-1002. The supernatants were centrifuged at 1500×g for 7 min to obtain cell-free samples. The samples were aliquoted and stored at −20° C. until further use. Production of IL-6 in the tissue culture supernatants was monitored using Quantikine® human MMP-3 (total) ELISA following the manufacturer's instructions. The data in FIG. 4 show that IDR-1002 alone did not stimulate production of IL-6. FLS cells stimulated with TGF-β produced more than 500 pg/ml of IL-6. However, stimulation of FLS cells with TGF-β in the presence of IDR-1002 resulted in a significant reduction, i.e., by more than 50%, in IL-6 production (FIG. 4). Results shown are an average of at least three independent biological experiments performed with cells isolated from synovial tissues obtained from independent donors±standard error (*p<0.05, **p<0.01).

### Example 5

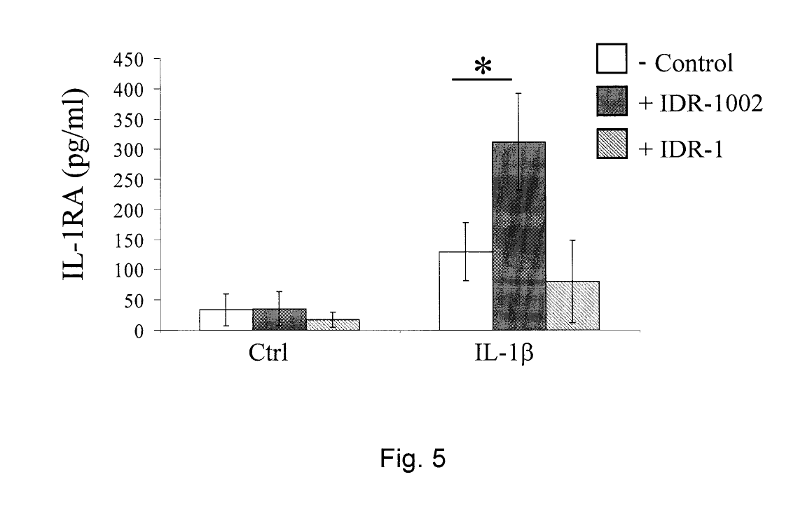

Tissue culture supernatants were harvested from human FLS cells after stimulation for 48 hr with IL-1β with and without IDR-1002. The supernatants were centrifuged at 1500×g for 7 min to obtain cell-free samples. The samples were aliquoted and stored at −20° C. until further use. Production of IL-IRA in the tissue culture supernatants was monitored using Quantikine® human MMP-3 (total) ELISA following the manufacturer's instructions. The data in FIG. 5 show that IDR-1002 alone did not stimulate production of IL-IRA. FLS cells stimulated with IL-1β produced about 150 pg/ml of IL-IRA, while FLS cells stimulated with IL-1β in the presence of IDR-1002 produced more than twice as much IL-IRA (FIG. 5). Furthermore, FLS cells stimulated with IL-1β in the presence of IDR-1002 produced more than twice as much IL-IRA produced by FLS cells stimulated with IL-1β in the presence of IDR-1 (FIG. 5). Results shown are an average of at least three independent biological experiments performed with cells isolated from synovial tissues obtained from independent donors±standard error (*p<0.05, **p<0.01).

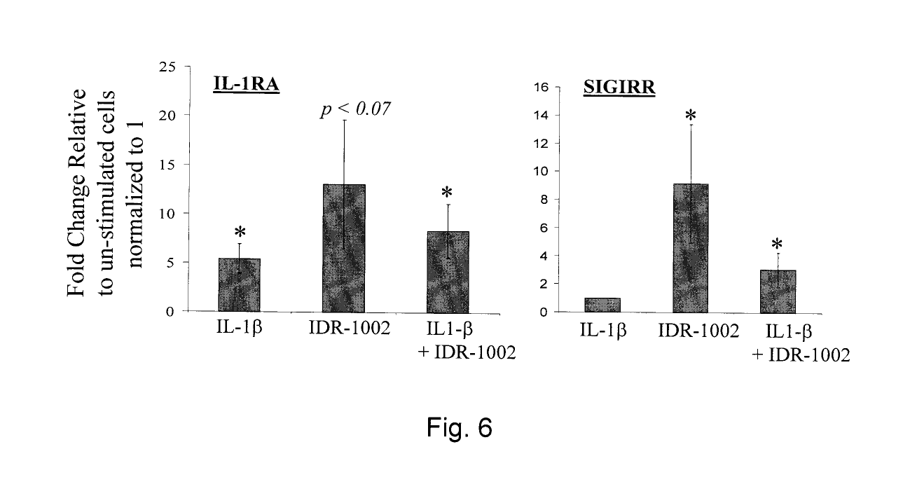

Additionally, transcriptional responses for IL-IRA and SIGIRR in these cell cultures were evaluated by quantitative real-time PCR after 2 hrs. The data in FIG. 6 show that IL-IRA transcription was significantly increased in cell cultures stimulated by: (i) IDR-1002 alone, and (ii) IDR-1002 plus IL-1β, in comparison to IL-1β alone. Also, SIGIRR transcription was significantly increased in cell cultures stimulated by: (i) IDR-1002 alone, and (ii) IDR-1002 plus IL-1β, in comparison to IL-1β alone (FIG. 6).

### Example 6

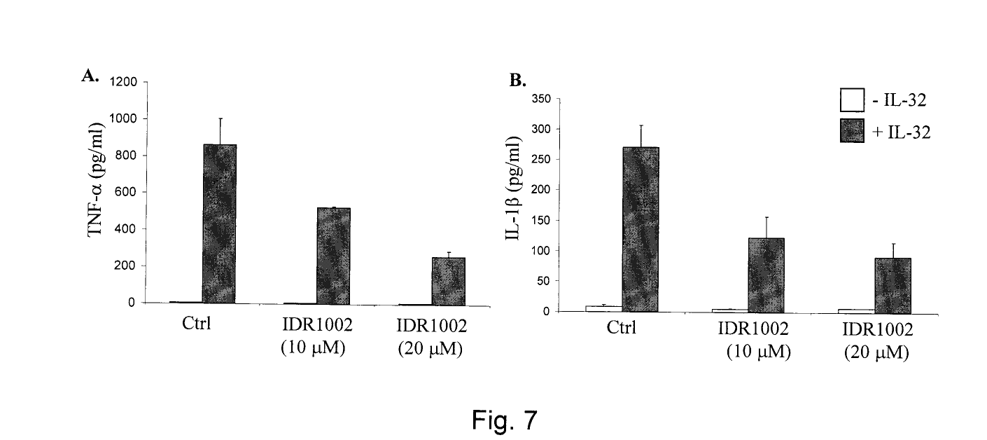

Human macrophage-like THP-1 cells were stimulated for 24 hr with pro-inflammatory cytokine IL-32 (20 ng/ml) in the presence and absence of IDR-1002 (10 or 20 μM). The tissue culture supernatants were monitored for the production of pro-inflammatory cytokines TNF-α and IL-1β after 24 hr by ELISA as described previously. The data in FIG. 7A show that the presence of 10 μM IDR-1002 during stimulation with IL-32 reduced TNF-α production by 40%, while the presence of 20 μM IDR-1002 reduced TNF-α production by about 70%. The data in FIG. 7B show that the presence of 10 μM IDR-1002 during stimulation with IL-32 reduced IL-1β production by about 40%, while the presence of 20 μM IDR-1002 reduced IL-1β production by about 65%.

### Example 7

Human FLS cells (5×10⁴/ml) were seeded in a total volume of 20 ml per 75 cm² tissue culture flask in complete DMEM media containing 10% FBS for each condition. The cells were allowed to adhere overnight. Following day the media was changed to 10 ml complete DMEM containing 1% FBS. The cells were either un-stimulated or treated with IL-1β (10 ng/ml) in the presence or absence of IDR-1002 (100 μg/ml) for 15 min. IL-1β is known to induce JNK activation, and also p38 MAPK activity, after 15 min in FLS cells. Peptide IDR-1002 (100 μg/ml) was added either 45 min prior to, or at the time of stimulation with IL-1β. Total protein concentration was evaluated for each cell lysate employing micro BCA (Thermo Scientific). Kinase activities specific to JNK and to p38-MAPK were monitored employing the JNK activity assay kit and the p38 MAPK assay kit (Abcam Inc., Calif., USA) following the manufacturer's instructions. For JNK, 50 μg of total protein per cell lysate was used for immunoprecipitation employing a JNK-specific antibody. The eluate was treated with c-Jun substrate and ATP mixture. Subsequent phosporylation of c-Jun was evaluated by probing immunoblots with anti-phospho-c-Jun (Ser73) specific antibody. For p38 MAPK, 50 μg of total protein per cell lysate was used for immunoprecipitation employing a p38-specific antibody. The eluate was treated with ATF-2 protein substrate and ATP mixture. Subsequent phosporylation of ATF-2 was evaluated by probing immunoblots with a phospho-ATF-2 (Thr76) specific antibody.

Total cell lysates were electrophoretically resolved on a 4-12% NuPAGE® Bis-Tris gels (NuPAGE is a registered trademark of the Invitrogen Corporation, Carlsbad, Calif., USA), followed by transfer to nitrocellulose membranes (Millipore, Canada). The membranes were subsequently probed with anti-phospho-c-Jun (Ser73) specific antibody (Abeam Inc.) in TBST (20 mM Tris pH 7.5, 150 mM NaCl, 0.1% Tween 20) containing 5% skimmed milk powder. Affinity purified HRP-linked anti-rabbit secondary antibody was used for detection. The membranes were development with Amersham ECL detection system (GE Healthcare, Baie d'Urfe QC, Canada) according to the manufacturer's instructions.

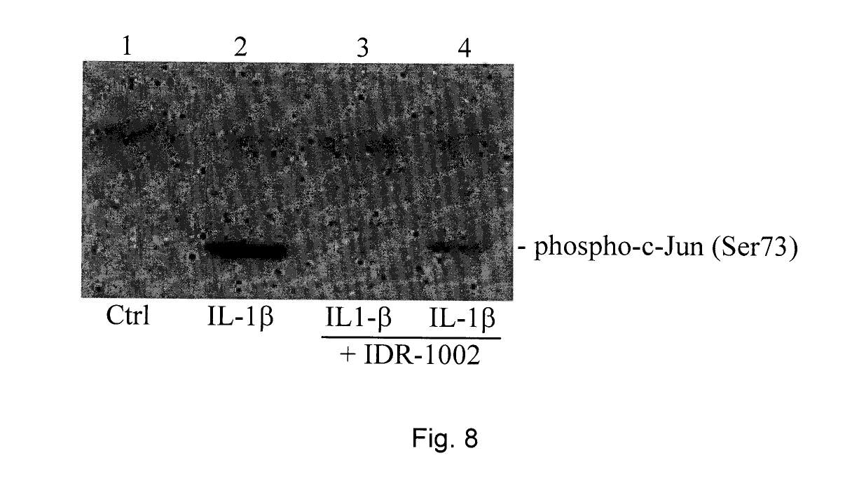

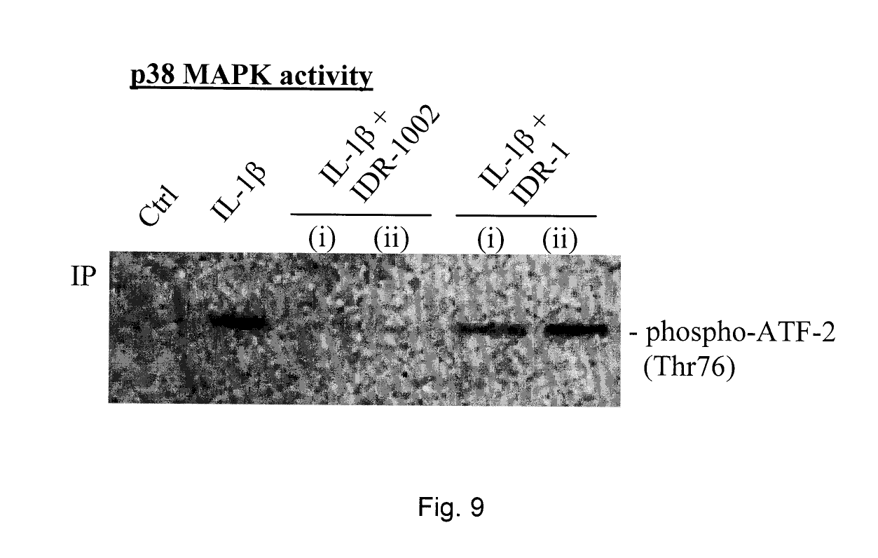

Samples were: (1) un-stimulated control, (2) IDR-1002 added 45 min prior to IL-1β stimulation, (3) IDR-1002 added simultaneously with IL-1β, and (4) IL-1β, were probed in immunoblots and represent at least three independent experiments using cells isolated from independent donors. IL-1β-induced JNK activity and consequently phophorylation of c-Jun (Ser73) was abrogated in the presence of the peptide IDR-1002 in human FLS cells (FIG. 8). IL-1β-induced p38 MAPK activity and consequently phophorylation of ATF-2 (Thr76) was abrogated in the presence of the peptide IDR-1002 in human FLS cells (FIG. 9).

We have shown that IDR-1002 can selectively modulate pro-inflammatory cytokine IL-1β-induced cellular responses in human FLS. We examined the effect of IDR-1002 on IL-1β-induced responses that contribute to tissue damage in inflammatory arthritis e.g. enzyme MMP-3 (stromelysin 1) and chemokine MCP-1. Both MMP-3 and MCP-1 are highly expressed in RA patients, known to promote inflammation within the synovial microenvironment and subsequent destruction of matrix components of the joints. IDR-1002 significantly suppressed IL-1β-induced MMP-3 and MCP-1 protein production in human FLS isolated from patients with inflammatory arthritis (FIGS. 1 and 2). The synergistically elevated level of MMP-3 protein in the presence of a combination of pro-inflammatory cytokines TNF-α and IL-1β was also significantly suppressed by IDR-1002 (FIG. 1). We also have shown that IDR-1002 suppressed TGF-β1-induced pro-inflammatory protein production in human FLS (FIG. 4). TGF-β1 contributes to the inflammatory pathogenesis of RA and induces mesenchymal transition/fibrosis in RA. Although IDR-1002 significantly suppressed pro-inflammatory responses, this peptide did not neutralize all chemokine production, e.g. IDR-1002 did not significantly suppress the expression of an anti-infective neutrophil chemokine IL-8 production in human FLS (FIG. 2C). However, unlike in macrophages, IDR-1002 did not induce chemokine production by itself in FLS, indicating chemokine induction by the peptide is cell type dependent. In contrast, IDR-1002 enhanced IL-1β-induced protein production of IL-1RA, which is an endogenous inhibitor of IL-1β (FIG. 5), and the peptide by itself up-regulated gene expression of IL-1RA in human FLS (FIG. 6). Similarly, we showed that the gene expression of another negative regulator of IL-1β, SIGIRR (single Ig IL-1R related molecule, also known as TIRE) was induced more than 9-fold by IDR-1002 relative to that observed in cells stimulated with IL-1β (FIG. 6). In addition to exhibiting selective anti-inflammatory effects in human FLS isolated from patients with inflammatory arthritis, we have also shown that IDR-1002 can inhibit pro-inflammatory responses e.g. TNF-α and IL-1β production, in human macrophages following stimulation with chronic inflammatory cytokine IL-32, which is elevated in inflammatory arthritis (FIG. 7). Taken together, we have demonstrated that IDR-1002 can selectively inhibit inflammatory responses both in immune cells such as macrophages, as well as localized structural cells such as mesenchymal human FLS, both critical cell types in the pathophysiology of RA.

As an approach to globally define the impact of IDR-1002 on IL-1β-induced protein production, we undertook a quantitative proteomic analysis. We demonstrated that IDR-1002 altered the IL-1β-induced proteome. Computational interrogation of IL-1β-induced proteins that were suppressed by IDR-1002, using a database that facilitates interaction analysis of mammalian immune genes and protein products, indicated that several members of NF-κB and MAPK-8 pathways were altered by IDR-1002. These included, (i) IκBκE; which activates NF-κB via TRAF-2, (ii) TRAF-6; a NF-κB regulator known to be critical in human autoimmune diseases including arthritis, (iii) TNF-receptor superfamily member TNFRSF21; which activates NF-κB and MAPK8 pathways, and (iv) NF-kappa-beta-inducing kinase (NIK); which activates NF-κB via TRAF-2, and (v) several members of the c-Jun N-terminal kinases of the JNK pathway, namely MAPK8, MAPK8IP1 and TNFRSF21. We confirmed these bioinformatics analyses using various immunochemical assays. We conclusively demonstrated that IDR-1002 abrogated IL-1β-induced JNK and p38 MAPK activity (FIG. 8), and significantly suppressed IL-1β-induced activation of NF-κB in synovial fibroblasts (FIG. 9). IL-1β-induced JNK and p38 MAPK activity is critical in the induction of MIMPs and tissue destruction in arthritis, therefore both JNK and p38 MAPK are valuable therapeutic targets for arthritis. Taken together, our results show that IDR-1002 suppressed IL-1β-induced MMP-3 and MCP-1 production, neutralized IL-1β-induced activation of JNK and p38 MAPK, and NF-κB activation, and suppressed TGF-1β-induced IL-6 in human FLS.

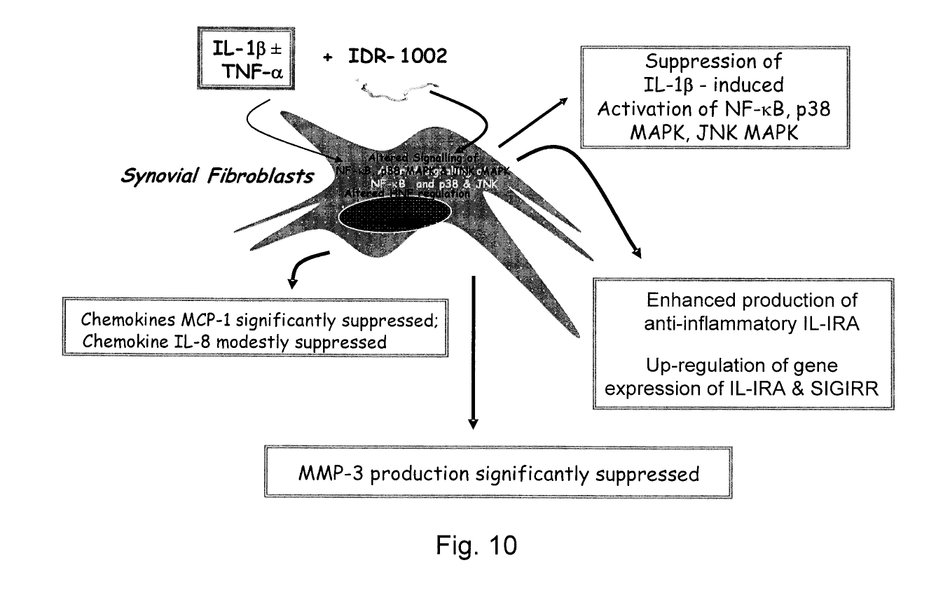

FIG. 10 shows a model summarizing the modulation of IL-1β-induced responses by IDR-1002 in synovial fibroblasts. Cellular uptake of peptide IDR-1002 may be mediated by unknown receptors or protein complexes. IDR-1002 results in the suppression of IL-1β-induced NF-κB, p38 MAPK activation and JNK MAPK activation. IDR-1002 also alters HNF-4α-mediated signalling and IL-1β-induced proteomes. Overall, IDR-1002 selectively suppresses downstream responses such as production of MMP-3 and MCP-1, but modestly impacts IL-8 production. Consequently, IDR-1002 suppresses cellular responses that lead to hyper inflammation and tissue destruction in arthritis.

1. An anti-inflammatory composition for prevention of or therapeutic treatment of arthritis in a subject, the composition comprising an effective amount of a polypeptide molecule that shares at least 80% sequence identity with an amino acid sequence selected from one of SEQ ID: 1 and SEQ ID NO: 2, and a pharmaceutically acceptable carrier.

2. An anti-inflammatory composition according to claim 1, wherein the polypeptide molecule comprises an amino acid sequence that shares at least 80% sequence identity with SEQ ID NO: 1.

3. An anti-inflammatory composition according to claim 1, wherein the polypeptide molecule comprises SEQ ID NO: 1.

4. An anti-inflammatory composition according to claim 1, wherein the polypeptide molecule comprises an amino acid sequence that shares at least 80% sequence identity with SEQ ID NO: 2.

5. An anti-inflammatory composition according to claim 1, wherein the polypeptide molecule comprises SEQ ID NO: 2.

6. Use of the composition of any of claims 1 to 5, for preventing the occurrence of arthritis symptoms in a subject in need thereof.

7. Use of the composition of any of claims 1 to 5, for therapeutic treatment of arthritis in a subject in need thereof.

8. Use of the composition of any of claims 1 to 5, for suppressing a cell-signalling pathway associated with predisposition of an arthritis condition or with an arthritis condition.

9. Use of the composition of any of claims 1 to 5, for suppressing an activation of or an expression of a pro-inflammatory cytokine.

10. Use according to claim 9, wherein the pro-inflammatory cytokine is interleukin-1 beta.

11. Use of the composition of any of claims 1 to 5, for suppressing an activation of or an expression of a matrix metallopeptidase-3.

12. A method of preventing or treating inflammatory arthritis comprising administering to a subject in need thereof, an effective amount of the composition according to any of claims 1 to 5.


SEQUENCE LISTING (3 sequences)

SEQ ID NO: 1 - protein, 12 aa, Artificial Sequence
  IDR-1002 peptide
VQRWLIVWRIRK

SEQ ID NO: 2 - protein, 15 aa, Artificial Sequence
  IDR-1002 peptide derivative
VQRWLIVWRIRKXAA

SEQ ID NO: 3 - protein, 13 aa, Artificial Sequence
  IDR-1 peptide
KSRIVPAIPVSLL